(19) United States
(12) Patent Application Publication
(10) Pub. No.: US 2020/0330496 A1
(43) Pub. Date: Oct. 22, 2020

(54) METHODS AND COMPOSITIONS FOR TREATING PREMATURE AGING DISEASES

(71) Applicant: Elysium Health, Inc., New York, NY (US)

(72) Inventors: Eric Marcotulli, New York, NY (US); Dan Alminana, New York, NY (US); Ryan Dellinger, Azusa, CA (US); Mark Morris, New York, NY (US)

(21) Appl. No.: 16/754,072

(22) PCT Filed: Oct. 5, 2018
(86) PCT No.: PCT/US18/54646

§ 371 Date: Apr. 6, 2020

Related U.S. Application Data
(60) Provisional application No. 62/568,950, filed on Oct. 6, 2017.

(51) Int. Cl. A61K 31/706; A61K 31/09; A61K 9/00; A61P 43/00

(52) U.S. Cl. CPC A61K 31/706; A61P 43/00; A61K 9/0053; A61K 31/09

(57) ABSTRACT

Provided herein are methods and compositions related to treating and/or preventing premature aging diseases and/or progeroid syndromes in a subject by administering to the subject (e.g., orally administering to the subject) a composition comprising nicotinamide riboside and/or pterostilbene.

### CROSS-REFERENCE TO RELATED APPLICATIONS

This application claims the benefit of U.S. Provisional Application No. 62/568950, filed on Oct. 6, 2017; hereby incorporated by reference in its entirety.

### BACKGROUND

Premature aging syndromes, such as progeria, are generally genetic disorders that clinically differ from the normal aging process. Individuals affected by these disorders can experience atrophy (skin thinning and loss of elasticity), loss of cutaneous fat, wrinkling, greying hair, loss of hair, nail dystrophy, defective pigmentation, and ulceration. These are changes that occur as the normal body ages, however, in premature aging disorders they occur at an accelerated rate.

Hutchinson-Gilford progeria syndrome and Werner syndrome are two of the best characterized human progeroid diseases with clinical features mimicking physiological aging at an early age. Most individuals Hutchinson-Gilford progeria syndrome and Werner syndrome will not survive past age thirty, and no specific treatment exists for any of these syndromes. Accordingly, there is a great need for new compositions and methods that treat diseases and disorders associated with premature aging.

### SUMMARY

Provided herein are methods and compositions related to treating diseases or disorders associated with premature aging, as well as for treating, preventing, and/or improving symptoms of a disease or disorder associated with premature aging by administering to a subject (e.g., orally administering to the subject) a composition comprising a compound of Formula I or Formula II (e.g., nicotinamide riboside), and/or a compound of Formula III (e.g., pterostilbene).

In certain aspects, the methods and compositions provided herein relate to treating progeria and/or a progeroid syndrome in a subject by administering to the subject (e.g., orally administering to the subject) a composition comprising a compound of Formula I or Formula II (e.g., nicotinamide riboside) and/or a compound of Formula III (e.g., pterostilbene). In certain aspects, the methods and compositions provided herein relate to treating, preventing or improving symptoms of a progeroid syndrome in a subject by administering to the subject (e.g., orally administering to the subject) a composition comprising a compound of Formula I or Formula II (e.g., nicotinamide riboside) and/or a compound of Formula III (e.g., pterostilbene).

In some embodiments, the disease or disorder associated with premature aging and/or the progeroid syndrome is a disease or disorder associated with or caused by a mutation in a gene encoding a lamin protein (e.g., lamin A and/or lamin C). In some embodiments, the disease or disorder associated with premature aging and/or a progeroid syndrome is a disease or disorder associated with or caused by a mutation in a gene encoding a DNA repair protein. Provided herein are methods of treating a disease or disorder associated with or caused by a mutation in a gene encoding for a DNA repair protein, comprising administering to the subject a composition comprising of Formula I or Formula II (e.g., nicotinamide riboside) and/or a compound of Formula III (e.g., pterostilbene). The DNA repair protein may be a RecQ protein-like helicases (RECQLs) or a nucleotide excision repair (NER) protein.

The disease or disorder associated with premature aging may be Hutchinson-Gilford progeria syndrome (HGPS), Werner syndrome (WS), Bloom syndrome (BS), Rothmund-Thomson syndrome (RTS), Cockayne syndrome (CS), xeroderma pigmentosum (XP), trichothiodystrophy (TTD), combined xeroderma pigmentosum-Cockayne syndrome (XP-CS), or restrictive dermopathy (RD). The progeroid syndrome may be Hutchinson-Gilford progeria syndrome (HGPS), Werner syndrome (WS), Rothmund-Thomson syndrome (RTS), Cockayne syndrome (CS), trichothiodystrophy (TTD), combined xeroderma pigmentosum-Cockayne syndrome (XP-CS), or restrictive dermopathy (RD).

In certain embodiments of the compositions and methods provided herein, the composition comprises a compound of Formula I or Formula II (e.g., nicotinamide riboside) (e.g., at least 100 mg, at least 125 mg, at least 150 mg, at least 175 mg, at least 200 mg, at least 225 mg, at least 250 mg, at least 275 mg, at least 300 mg, at least 325 mg, at least 350 mg, at least 375 mg, at least 400 mg, at least 425 mg, at least 450 mg, at least 475 mg, at least 500 mg, at least 525 mg, at least 550 mg, at least 575 mg or at least 600 mg of a compound of Formula I or Formula II (e.g., nicotinamide riboside)). In some embodiments, the composition comprises a compound of Formula III (e.g., pterostilbene) (e.g., at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 125 mg or at least 150 mg of a compound of Formula III (e.g., pterostilbene)). In certain embodiments, the composition comprises both a compound of Formula I or Formula II (e.g., nicotinamide riboside) (e.g., at least 100 mg, at least 125 mg, at least 150 mg, at least 175 mg, at least 200 mg, at least 225 mg, at least 250 mg, at least 275 mg, at least 300 mg, at least 325 mg, at least 350 mg, at least 375 mg, at least 400 mg, at least 425 mg, at least 450 mg, at least 475 mg, at least 500 mg, at least 525 mg, at least 550 mg, at least 575 mg or at least 600 mg of a compound of Formula I or Formula II (e.g., nicotinamide riboside)) and a compound of Formula III (e.g., pterostilbene) (e.g., at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 125 mg or at least 150 mg of a compound of Formula III (e.g., pterostilbene)).

In certain embodiments, the method comprises administering a plurality of doses of the composition. In some embodiments, at least 7 doses of the composition are administered. In some embodiments, at least 30 doses of the composition are administered. In some embodiments, at least 60 or more doses of the composition are administered. In some embodiments, each dose comprises at least 100 mg, at least 125 mg, at least 150 mg, at least 175 mg, at least 200 mg, at least 225 mg, at least 250 mg, at least 275 mg, at least 300 mg, at least 325 mg, at least 350 mg, at least 375 mg, at least 400 mg, at least 425 mg, at least 450 mg, at least 475 mg, at least 500 mg, at least 525 mg, at least 550 mg, at least 575 mg or at least 600 mg of a compound of Formula I or Formula II (e.g., nicotinamide riboside). In some embodiments, each dose comprises at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 125 mg or at least 150 mg of a compound of Formula III (e.g., pterostilbene). In certain embodiments, each dose comprises at least 100 mg, at least 125 mg, at least 150 mg, at least 175 mg, at least 200 mg, at least 225 mg, at least 250 mg, at least 275 mg, at least 300 mg, at least 325 mg, at least 350 mg, at least 375 mg, at least 400 mg, at least 425 mg, at least 450 mg, at least 475 mg, at least 500 mg, at least 525 mg, at least 550 mg, at least 575 mg or at least 600 mg of a compound of Formula I or Formula II (e.g., nicotinamide riboside) at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 125 mg or at least 150 mg of a compound of formula III (e.g., pterostilbene).

In certain embodiments, a dose of the composition is administered at regular intervals over a period of time. In some embodiments, a dose of the composition is administered at least once a week. In some embodiments, a dose of the composition is administered at least twice a week. In certain embodiments, a dose of the composition is administered at least three times a week. In some embodiments, a dose of the composition is administered at least once a day. In some embodiments, a dose of the composition is administered at least twice a day. In some embodiments, doses of the composition are administered for at least 1 week, for at least 2 weeks, for at least 3 weeks, for at least 4 weeks, for at least 1 month, for at least 2 months, for at least 3 months, for at least 4 months, for at least 5 months, for at least 6 months or for at least 1 year.

In certain embodiments, the composition is formulated for oral delivery. In some embodiments, the composition is formulated as a pill, a tablet, or a capsule. In some embodiments, the composition is administered orally. In certain embodiments, the composition is self-administered.

### DETAILED DESCRIPTION

### General

Provided herein are methods and compositions related to treating a disease or disorder associated with premature aging, such as a progeroid syndrome, in a subject by administering to the subject (e.g., orally administering to the subject) a composition comprising a compound of Formula I or Formula II (e.g., nicotinamide riboside) and/or a compound of Formula III (e.g., pterostilbene). In some aspects, provided herein are methods and compositions related to treating, preventing, and/or improving the symptoms of a disorder associated with premature aging, such as a progeroid syndrome, in a subject by administering to the subject (e.g., orally administering to the subject) a composition comprising a compound of Formula I or Formula II (e.g., nicotinamide riboside) and/or a compound of Formula III (e.g., pterostilbene).

The progeroid syndrome may be progeria. In certain aspects, provided herein are methods and compositions related to treating or treating a disease or disorder associated with mutations in a gene encoding a lamin protein (e.g., lamin A and/or lamin C). In other aspects, provided herein are methods and compositions related to treating or treating a disease or disorder associated with mutations in a gene encoding a DNA repair protein.

### Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

As used herein, the term “administering” means providing a pharmaceutical agent or composition to a subject, and includes, but is not limited to, administering by a medical professional and self-administering. Administration of a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

The phrase “pharmaceutically-acceptable carrier” as used herein means a pharmaceutically-acceptable material, composition or vehicle, such as a liquid or solid filler, diluent, excipient, or solvent encapsulating material.

As used herein, the term “subject” means a human or non-human animal selected for treatment or therapy.

The phrases “therapeutically-effective amount” and “effective amount” as used herein means the amount of an agent which is effective for producing the desired therapeutic effect in at least a sub-population of cells in a subject at a reasonable benefit/risk ratio applicable to any medical treatment.

“Treating” a disease in a subject or “treating” a subject having a disease refers to subjecting the subject to a pharmaceutical treatment, e.g., the administration of a drug, such that at least one symptom of the disease is decreased or prevented from worsening.

As used herein, a therapeutic that “prevents” a disorder or condition refers to a compound that, when administered to a statistical sample prior to the onset of the disorder or condition, reduces the occurrence of the disorder or condition in the treated sample relative to an untreated control sample, or delays the onset or reduces the severity of one or more symptoms of the disorder or condition relative to the untreated control sample.

### Compositions

Provided herein are pharmaceutical compositions comprising a compound of Formula I or Formula II (e.g., nicotinamide riboside) and/or a compound of Formula III (e.g., pterostilbene).

Nicotinamide riboside is a pyridine-nucleoside form of niacin (i.e., vitamin B3) that serves as a precursor to nicotinamide adenine dinucleotide (NAD⁺). As used herein, “nicotinamide riboside” also includes nicotinamide riboside salts, such as nicotinamide riboside chloride. The chemical structure of nicotinamide riboside is provided below:

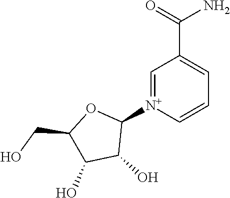 [SMILES: NC(=O)c1ccc[n+]([C@@H]2O[C@H](CO)[C@@H](O)[C@H]2O)c1]

In some embodiments, provided herein are pharmaceutical compositions comprising a compound represented by Formula (I) or a pharmaceutically acceptable salt thereof:

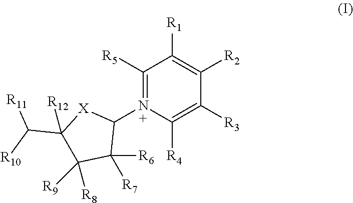

wherein, independently for each occurrence:

R₁, R₂, and R₃ are selected from hydrogen, halogen, —CN, —NO₂, —OR₁₄, —N(R₁₄)ₘ, —R₁₃, substituted or unsubstituted (C₁-C₆)alkyl, cycloalkyl, heterocycloalkyl, aryl, heteroaryl, aralkyl, and heteroaralkyl;

R₄ and R₅ are selected from hydrogen, halogen, —CN, —NO₂, —OR₁₄, —N(R₁₄)ₘ, substituted or unsubstituted (C₁-C₆)alkyl, cycloalkyl, heterocycloalkyl, aryl, heteroaryl, aralkyl, and heteroaralkyl;

R₆, R₈, R₁₁, and R₁₂ are selected from hydrogen, (C₁-C₆)alkyl, C₆)alkylene)N(R₁₄)ₘ, —C(O)((C₁-C₆)alkylene)N(R₁₄)ₘ, cycloalkyl, heterocycloalkyl, aryl, heteroaryl, aralkyl, heteroaralkyl, —OR₁₄, and —N(R₁₄)ₘ;

R₇, R₉, and R₁₀ are selected from —((C₁-C₆)alkylene)N(R₁₄)ₘ, —OR₁₄, and —N(R₁₄)ₘ;

R₁₃ is selected from —OR₁₄, —N(R₁₄)ₘ, —C(O)(R₁₄), —C(O)(OR₁₄), —C(O)N(R₁₄)ₘ, —S(O)₂(OR₁₄), —S(O)OR₁₄, and —S(O)₂N(R₁₄)ₘ;

R₁₄ is selected from hydrogen, (C₁-C₆)alkyl, cycloalkyl, heterocycloalkyl, aryl, heteroaryl, aralkyl, and heteroaralkyl; and

X is O, S, or N(R₁₄);

m is 2 or 3;

provided that at least one of R₁, R₂, and R₃ is R₁₃.

In some embodiments, R₁ is R₁₃. In some embodiments, R₂ is R₁₃. In some embodiments, R₃ is R₁₃.

In some embodiments, R₁₃ is selected from —OR₁₄, —N(R₁₄)ₘ, —C(O)(R₁₄), —C(O)(OR₁₄), and —C(O)N(R₁₄)ₘ. In some embodiments, R₁₃ is selected from —C(O)(R₁₄), —C(O)(OR₁₄), and -C(O)N(R₁₄)ₘ. In some embodiments, R₁₃ is -C(O)N(R₁₄)ₘ.

In some embodiments, R₇, R₉, and R₁₀ are each independently —OR₁₄ or —N(R₁₄)ₘ. In some embodiments, R₇, R₉, and R₁₀ are —OR₁₄.

In some embodiments, the compound of formula (I) is represented by Formula (II) or a pharmaceutically acceptable salt thereof:

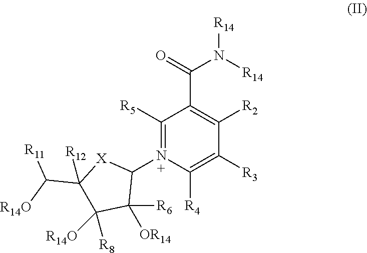

wherein, independently for each occurrence:

R₂ and R₃ are selected from hydrogen, halogen, —CN, —NO₂, —OR₁₄, —N(R₁₄)ₘ, —R₁₃, substituted or unsubstituted (C₁-C₆)alkyl, cycloalkyl, heterocycloalkyl, aryl, heteroaryl, aralkyl, and heteroaralkyl;

R₄ and R₅ are selected from hydrogen, halogen, —CN, —NO₂, —OR₁₄, —N(R₁₄)ₘ, substituted or unsubstituted (C₁-C₆)alkyl, cycloalkyl, heterocycloalkyl, aryl, heteroaryl, aralkyl, and heteroaralkyl;

R₅, R₈, R₁₁, and R₁₂ are selected from hydrogen, —OR₁₄, —N(R₁₄)ₘ, substituted or unsubstituted (C₁-C₆)alkyl, —((C₁-C₆)alkylene)N(R₁₄)ₘ, —C(O)((C₁-C₆)alkylene)N(R₁₄)ₘ, cycloalkyl, heterocycloalkyl, aryl, heteroaryl, aralkyl, and heteroaralkyl;

R₁₃ is selected from —OR₁₄, —N(R₁₄)ₘ, —C(O)(R₁₄), —C(O)(OR₁₄), —C(O)N(R₁₄)ₘ, —S(O)₂(OR₁₄), —S(O)OR₁₄, and —S(O)₂N(R₁₄)ₘ;

R₁₄ is selected from hydrogen, (C₁-C₆)alkyl, cycloalkyl, heterocycloalkyl, aryl, heteroaryl, aralkyl, and heteroaralkyl; and

m is 2 or 3.

In some embodiments of the compounds of formula (I) or (II), R₁, R₂, and R₃ are each independently, if present, selected from hydrogen, halogen, —CN, —NO₂, —OR₁₄, —N(R₁₄)ₘ, —R₁₃, and substituted or unsubstituted (C₁-C₆)alkyl. In some embodiments, R₁, R₂, and R₃ are each independently, if present, selected from hydrogen, —OR₁₄, —N(R₁₄)ₘ, and unsubstituted (C₁-C₆)alkyl. In some embodiments, R₁, R₂, and R₃ are each independently, if present, selected from substituted or unsubstituted (C₁-C₆)alkyl, cycloalkyl, heterocycloalkyl, aryl, heteroaryl, aralkyl, and heteroaralkyl. In some embodiments, R₁, R₂, and R₃ are each independently, if present, hydrogen.

In some embodiments of the compounds of formula (I) or (II), R₄ and R₅ are each independently selected from hydrogen, halogen, —CN, —NO₂, —OR₁₄, —N(R₁₄)ₘ, and substituted or unsubstituted (C₁-C₆)alkyl. In some embodiments, R₄ and R₅ are each independently selected from hydrogen, —OR₁₄, —N(R₁₄)ₘ, and unsubstituted (C₁-C₆)alkyl. In some embodiments, R₄ and R₅ are each independently selected from substituted or unsubstituted (C₁-C₆)alkyl, cycloalkyl, heterocycloalkyl, aryl, heteroaryl, aralkyl, and heteroaralkyl. In some embodiments, R₄ and R₅ are each hydrogen.

In some embodiments of the compounds of formula (I) or (II), R₆, R₈, R₁₁, and R₁₂ are selected from hydrogen, —OR₁₄, —N(R₁₄)ₘ, unsubstituted (C₁-C₆)alkyl, —((C₁-C₆)alkylene)N(R₁₄)ₘ, —C(O)((C₁-C₆)alkylene)N(R₁₄)ₘ, cycloalkyl, heterocycloalkyl, aryl, heteroaryl, aralkyl, and heteroaralkyl. In some embodiments, R₆, R₈, R₁₁, and R₁₂ are each independently selected from hydrogen, —OR₁₄, —N(R₁₄)ₘ, unsubstituted (C₁-C₆)alkyl, —((C₁-C₆)alkylene)N(R₁₄)ₘ, and —C(O)((C₁-C₆)alkylene)N(R₁₄)ₘ. In some embodiments, R₆, R₈, R₁₁, and R₁₂ are each independently selected from hydrogen, —OR₁₄, and —N(R₁₄)ₘ. In some embodiments, R₆, R₈, R₁₁, and R₁₂ are each independently selected from unsubstituted (C₁-C₆)alkyl, cycloalkyl, heterocycloalkyl, aryl, heteroaryl, aralkyl, and heteroaralkyl. In some embodiments, R₆, R₈, R₁₁, and R₁₂ are each hydrogen.

In some embodiments, R₇, R₉, and R₁₀ are each independently —OR₁₄ or —N(R₁₄)ₘ. In some embodiments, R₇, R. and R₁₀ are each —OR₁₄. In some embodiments, R₇, R₉, and R₁₀ are each —OH.

In some embodiments of the compounds of formula (I) or (II), R₁₄ is hydrogen or (C₁-C₆)alkyl.

In some embodiments of the compounds of formula (I) or (II), X is O or N(R₁₄). In some embodiments, X is O.

In some embodiments of the compounds of formula (I) or (II), the compound is

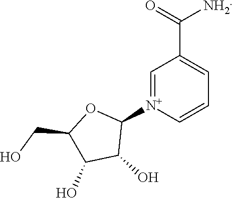 [SMILES: NC(=O)c1ccc[n+]([C@@H]2O[C@H](CO)[C@@H](O)[C@H]2O)c1]

Pterostilbene is a stilbenoid and an analog of polyphenol reservatrol that has better bioavailability due to the presence of two methoxy groups that allow it to have increased lipophilic and oral absorption as well as a longer half-life due to reduced oxidation. The chemical structure of pterostilbene is provided below:

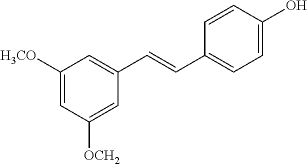

In some embodiments, provided herein are pharmaceutical compositions comprising a compound represented by Formula (III) or a pharmaceutically acceptable salt thereof:

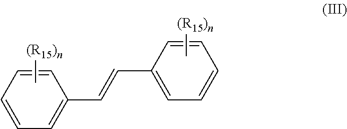

wherein, independently for each occurrence:

R₁₃ is selected from halogen, —CN, —NO₂, —OR₁₆, —N(R₁₆)ₚ, —S(O)₂(OR₁₆), —S(O)OR₁₆, substituted or unsubstituted (C₁-C₆)alkyl, cycloalkyl, heterocycloalkyl, aryl, heteroaryl, aralkyl, and heteroaralkyl;

R₁₆ is selected from hydrogen, (C₁-C₆)alkyl, cycloalkyl, heterocycloalkyl, aryl, heteroaryl, aralkyl, and heteroaralkyl;

n is an integer from 0 to 5; and

p is 2 or 3;

provided that at least one n is 1; and at least one R₁₃ is —OR₁₆;

provided that the compound of formula (III) is not

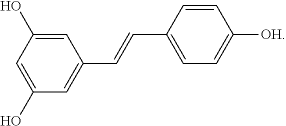 [SMILES: Oc1ccc(/C=C/c2cc(O)cc(O)c2)cc1]

In some embodiments of the compounds of formula (III), R₁₅ is selected from, halogen, —CN, —NO₂, —OR₁₆, —N(R₁₆)ₚ, and substituted or unsubstituted (C₁-C₆)alkyl. In some embodiments, R₁₅ is selected from —OR₁₆, —N(R₁₆)ₚ, and unsubstituted (C₁-C₆)alkyl. In some embodiments, R₁₅ is selected from substituted or unsubstituted (C₁-C₆)alkyl, cycloalkyl, heterocycloalkyl, aryl, heteroaryl, aralkyl, and heteroaralkyl. In some embodiments, R₁₅ is —OR₁₆. In some embodiments, R₁₅ is —OR₁₆; and R₁₆ is hydrogen or (C₁-C₆)alkyl. In some embodiments, R₁₅ is —OR_{16;} and R₁₆ is (C₁-C₆)alkyl, cycloalkyl, heterocycloalkyl, aryl, heteroaryl, aralkyl, and heteroaralkyl. In some embodiments, R₁₅ is —OR₁₆; and R₁₆ is (C₁-C₆)alkyl. In some embodiments, R₁₅ is —OR₁₆; and R₁₆ is (C₁-C₆)alkyl, cycloalkyl, or heterocycloalkyl.

In some embodiments, n is 1, 2, or 3. In some embodiments, n is 1 or 2.

In some embodiments, p is 2. In some embodiments, p is 3.

In one aspect, the provided herein are pharmaceutically acceptable compositions which comprise a therapeutically-effective amount of one or more of the compounds described herein (e.g., nicotinamide riboside and/or pterostilbene), formulated together with one or more pharmaceutically acceptable carriers (additives) and/or diluents. In another aspect, the agents described herein can be administered as such, or administered in mixtures with pharmaceutically acceptable carriers and can also be administered in conjunction with other agents. Conjunctive therapy thus includes sequential, simultaneous and separate, or co-administration of one or more compounds of the invention, wherein the therapeutic effects of the first administered has not entirely disappeared when the subsequent compound is administered.

As described in detail below, the pharmaceutical compositions described herein may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; or (3) sublingually.

In some embodiments, the composition comprises additional agents. For example, the composition may comprise a nutritional agent, such as an antioxidant. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The formulations of the compounds described herein may be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient which can be combined with a carrier material to produce a single dosage form will generally be that amount of the agent which produces a therapeutic effect.

In certain embodiments, a formulation described herein comprises an excipient, including, but not limited to, cyclodextrins, liposomes, micelle forming agents, e.g., bile acids, and polymeric carriers, e.g., polyesters and polyanhydrides; and an agent of the invention. In some embodiments, an aforementioned formulation renders orally bioavailable an agent of the invention. Methods of preparing these formulations or compositions may include the step of bringing into association a compound of the invention with the carrier and, optionally, one or more accessory ingredients.

Liquid dosage forms for oral administration of the formulations provided herein include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations provided herein suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the invention as an active ingredient. A compound of the invention may also be administered as a bolus, electuary, or paste.

In solid dosage forms of the invention for oral administration (e.g., capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol, glycerol monostearate, and non-ionic surfactants; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions described herein, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. Compositions described herein may also be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Pharmaceutical compositions provided herein suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

### Therapeutic Methods

Provided herein are methods and compositions related to treating diseases or disorders associated with premature aging and for treating, preventing, or improving the symptoms of diseases or disorders associated with premature aging in a subject by administering to the subject (e.g., orally administering to the subject) a composition comprising a compound of Formula I or Formula II (e.g., nicotinamide riboside), and/or a compound of Formula III (e.g., pterostilbene). Examples of premature aging disease or disorders include, but are not limited to, is Hutchinson-Gilford progeria syndrome (HGPS), Werner syndrome (WS), Bloom syndrome (BS), Rothmund-Thomson syndrome (RTS), Cockayne syndrome (CS), xeroderma pigmentosum (XP), trichothiodystrophy (TTD), combined xeroderma pigmentosum-Cockayne syndrome (XP-CS), or restrictive dermopathy (RD). In some embodiments, the disease or disorder associated with premature aging is a progeroid syndrome.

Provided herein are methods and compositions related to treating a progeroid syndrome in a subject by administering to the subject (e.g., orally administering to the subject) a composition comprising a compound of Formula I or Formula II (e.g., nicotinamide riboside), and/or a compound of Formula III (e.g., pterostilbene). The subject may be male or female. In some embodiments, the subject is an adult (i.e., 18 years of age or older). The subject may be pediatric (i.e., less than 18 years of age). In some embodiments, the subject is a mammal, preferably, a human.

In some embodiments, the progeroid syndrome is progeria. Examples of progeroid syndromes include, but are not limited to, are Hutchinson-Gilford progeria syndrome (HGPS), Werner syndrome (WS), Rothmund-Thomson syndrome (RTS), Cockayne syndrome (CS), trichothiodystrophy (TTD), combined xeroderma pigmentosum-Cockayne syndrome (XP-CS), or restrictive dermopathy (RD).

The disease or disorder associated with premature ageing may be associated with or caused by a mutation in a gene encoding a lamin protein. In some embodiments, provided herein are methods of treating a disease or disorder associated with or caused by a mutation in a gene encoding a lamin protein (e.g., lamin A or lamin C) in a subject by administering to the subject (e.g., orally administering to the subject) a composition comprising a compound of Formula I or Formula II (e.g., nicotinamide riboside), and/or a compound of Formula III (e.g., pterostilbene). The disease or disorder associated with or caused by a mutation in a lamin protein may be Hutchinson-Gilford progeria syndrome (HGPS). The disease or disorder associated with or caused by a mutation in a lamin protein may be restrictive dermopathy (RD).

Products of the LMNA gene, primarily lamin A and C, are key components of the nuclear lamina, a proteinaceous meshwork that underlies the inner nuclear membrane and is essential for proper nuclear architecture. Alterations in lamin A and C that disrupt the integrity of the nuclear lamina affect a whole repertoire of nuclear functions, causing cellular decline. In humans, mutations in the LMNA gene have been identified and correlated with degenerative disorders. These disorders are referred to as laminopathies, and they include premature aging diseases. Hutchinson-Gilford Progeria Syndrome (HGPS) is a premature aging disease and laminpathy, caused by aberrant splicing of the LMNA gene and expression of a mutant product called progerin. Progerin accumulation elicits nuclear morphological abnormalities, misregulated gene expression, defects in DNA repair, telomere shortening, and genomic instability, all of which limit cellular proliferative capacity. In patients harboring this mutation, a severe premature aging disease develops during childhood. Signs and symptoms vary in age of onset and severity. Individuals with Hutchinson-Gilford progeria syndrome (HGPS) usually appear normal at birth. Profound failure to thrive occurs during the first year, and characteristic facies, with receding mandible, narrow nasal bridge and pointed nasal tip develop. During the first to third year, the following usually become apparent: partial alopecia progressing to total alopecia, loss of subcutaneous fat, progressive joint contractures, bone changes, nail dystrophy, and abnormal tightness and/or small soft outpouchings of the skin over the abdomen and upper thighs, and delayed primary tooth eruption. Later findings include low-frequency conductive hearing loss, dental crowding, and partial lack of secondary tooth eruption. Additional findings present in some but not all affected individuals include photophobia, excessive ocular tearing, exposure keratitis, and Raynaud phenomenon. Motor and mental development is normal. Death occurs as a result of complications of severe atherosclerosis, either cardiac disease (myocardial infarction) or cerebrovascular disease (stroke), generally between ages six and 20 years. In some embodiments, provided herein are methods of treating HGPS by administering to the subject (e.g., orally administering to the subject) a composition comprising a compound of Formula I or Formula II (e.g., nicotinamide riboside), and/or a compound of Formula III (e.g., pterostilbene).

The disease or disorder associated with premature ageing may be associated with or caused by a mutation in a gene encoding a DNA repair protein. Another cause of premature aging diseases and progeroid syndromes are genetic mutations which lead to defects in the cellular processes which repair DNA. The DNA damage theory of aging proposes that aging is a consequence of the accumulation of naturally occurring DNA damages. The accumulated damage may arise from reactive oxygen species (ROS), chemical reactions (e.g. with intercalating agents), radiation, depurination, and deamination. Mutations in DNA repair proteins, including RecQ protein-like helicases (RECQLs) and nucleotide excision repair (NER) proteins have been associated with diseases and disorders associated with premature aging and progeroid syndromes. In some aspects, provided herein are methods of treating a disease or disorder associated with or caused by a mutation in gene encoding a DNA repair protein, comprising administering to the subject a composition comprising of Formula I or Formula II (e.g., nicotinamide riboside) and/or a compound of Formula III (e.g., pterostilbene). The DNA repair protein may be RecQ protein-like helicases (RECQLs) or a nucleotide excision repair (NER) protein.

In some embodiments, the disease or disorder associated with premature aging may be caused by a mutation in a gene encoding a RecQ protein. In some embodiments, the progeroid syndrome may be a RecQ-associated progeroid syndrome. RecQ is a family of conserved ATP-dependent helicases required for repairing DNA and preventing deleterious recombination and genomic instability. There are five genes encoding RecQ in humans (RECQ1-5), and defects in RECQL2/WRN, RECQL3/BLM and RECQL4 lead to Werner syndrome (WS), Bloom syndrome (BS), and Rothmund-Thomson syndrome (RTS), respectively. On the cellular level, cells of affected individuals exhibit chromosomal abnormalities and genomic instability. Affected individuals may exhibit symptoms of growth retardation, short stature, premature graying of hair, hair loss, wrinkling, prematurely aged faces, beaked noses, skin atrophy (wasting away) with scleroderma-like lesions, loss of fat tissues, abnormal fat deposition leading to thin legs and arms, and severe ulcerations around the Achilles tendon and malleoli. Other symptoms include change in voice, making it weak, hoarse, or high-pitched; atrophy of gonads, leading to reduced fertility; bilateral cataracts (clouding of lens); premature arteriosclerosis (thickening and loss of elasticity of arteries); calcinosis (calcium deposits in blood vessels); atherosclerosis (blockage of blood vessels); type 2 diabetes; loss of bone mass; telangiectasia; and malignancies.

In some embodiments, the disease or disorder related to premature aging may be associated with or caused by a mutation in a gene encoding a NER protein. In some embodiments, the progeroid syndrome may be a NER-associated progeroid syndrome. Nucleotide excision repair (NER) is a DNA repair mechanism. In NER, the damaged DNA strand is removed and the undamaged strand is kept as a template for the formation of a complementary sequence with DNA polymerase. Examples of NER-associated syndromes include Cockayne syndrome, xenoderma pigmentosum, and trichothiodystrophy. Examples of NER-associated progeroid syndromes include Cockayne syndrome and trichothiodystrophy. Individuals affected by a NER-associated progeroid disorder or a disorder caused by a mutation in a gene encoding for a NER DNA repair protein include, but are not limited to, small head size (microcephaly), a failure to gain weight and grow at the expected rate (failure to thrive) leading to very short stature, and delayed development. The signs and symptoms of this condition are usually apparent from infancy, and they worsen over time. Most affected individuals have an increased sensitivity to sunlight (photosensitivity), and in some cases even a small amount of sun exposure can cause a sunburn or blistering of the skin. Other signs and symptoms often include hearing loss, vision loss, severe tooth decay, and bone abnormalities.

In some embodiments, provided herein are methods of treating Cockayne syndrome by administering to the subject (e.g., orally administering to the subject) a composition comprising a compound of Formula I or Formula II (e.g., nicotinamide riboside), and/or a compound of Formula III (e.g., pterostilbene).

In some embodiments, provided herein are methods of treating Werner Syndrome by administering to the subject (e.g., orally administering to the subject) a composition comprising a compound of Formula I or Formula II (e.g., nicotinamide riboside), and/or a compound of Formula III (e.g., pterostilbene).

Additional examples of progeroid syndromes include Marfan-progeroid-lipodystrophy syndrome. Patients with Marfan-progeroid-lipodystrophy syndrome typically exhibit congenital lipodystrophy and a neonatal progeroid appearance. The condition is caused by mutations near the 3′-terminus of the FBN1 gene. In some embodiments, the progeroid syndrome is Marfan-progeroid-lipodystrophy syndrome. In some embodiments, the progeroid syndrome is associated with or caused by a pathogenic mutation in the FBN1 gene.

In some embodiments, provided herein are methods of treating, improving, and/or preventing the symptoms of a premature aging disease (e.g., a premature aging disease disclosed herein) and/or a progeroid disorder (e.g., a progeroid disorder disclosed herein) by administering to the subject (e.g., orally administering to the subject) a composition comprising a compound of Formula I or Formula II (e.g., nicotinamide riboside), and/or a compound of Formula III (e.g., pterostilbene).

In some embodiments, provided herein are methods of treating and/or preventing the symptoms of HGPS by administering to the subject (e.g., orally administering to the subject) a composition comprising a compound of Formula I or Formula II (e.g., nicotinamide riboside), and/or a compound of Formula III (e.g., pterostilbene).

In some embodiments, provided herein are methods of treating and/or preventing the symptoms of Cockayne syndrome by administering to the subject (e.g., orally administering to the subject) a composition comprising a compound of Formula I or Formula II (e.g., nicotinamide riboside), and/or a compound of Formula III (e.g., pterostilbene).

In some embodiments, provided herein are methods of treating and/or preventing the symptoms of Werner Syndrome by administering to the subject (e.g., orally administering to the subject) a composition comprising a compound of Formula I or Formula II (e.g., nicotinamide riboside), and/or a compound of Formula III (e.g., pterostilbene).

Actual dosage levels and administration regimen of the compositions disclosed herein may be varied so as to obtain an amount of a compound of Formula I or Formula II (e.g., nicotinamide riboside) and/or a compound of Formula III (e.g., pterostilbene) that is effective to achieve the desired therapeutic response for a particular patient, composition, and mode of administration, without being toxic to the patient. In some embodiments, the subject continuously self-administers the compounds disclosed herein. In other embodiments, the subject may take a compound disclosed herein as needed.

In some embodiments, administration of the composition comprises administration of the composition in one or more dose(s). In some embodiments, administration of the composition comprises administration of the composition in one or more, five or more, ten or more, twenty or more, thirty or more, forty or more, fifty or more, one hundred or more, or one thousand or more dose(s). In some embodiments, the dose comprises at least 25 mg, at least 50 mg, at least 75 mg, at least 100 mg, at least 125 mg, at least 150 mg, at least 200 mg, at least 225 mg, at least 250 mg, at least 275 mg, at least 300 mg, at least 325 mg, at least 350 mg, at least 375 mg, at least 400 mg, at least 425 mg, at least 450 mg, at least 475 mg, at least 500 mg, at least 550 mg, at least 600 mg, at least 650 mg, at least 700 mg, at least 750 mg, at least 800 mg, or at least 850 mg of a compound of Formula I or Formula II (e.g., nicotinamide riboside). In some embodiments, the dose comprises at least 5 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 90 mg, at least 95 mg, at least 100 mg, at least 110 mg, at least 120 mg, at least 130 mg, at least 140 mg, at least 150 mg, at least 160 mg, least 170 mg, at least 180 mg, at least 190 mg, at least 200 mg, or at least 250 mg of a compound of formula III (e.g., pterostilbene) .

The compositions disclosed herein may be administered over any period of time effective to achieve the desired therapeutic response for a particular patient, composition, and mode of administration, without being toxic to the patient. The period of time may be at least 1 day, at least 10 days, at least 20 days, at least 30, days, at least 60 days, at least three months, at least six months, at least a year, at least three years, at least five years, or at least ten years. The dose may be administered when needed, sporadically, or at regular intervals. For example, the dose may be administered monthly, weekly, biweekly, triweekly, once a day, or twice a day.

### Incorporation by Reference

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### Equivalents

Those skilled in the art will recognize, or be able to ascertain using no more than routine experimentation, many equivalents to the specific embodiments of the invention described herein. Such equivalents are intended to be encompassed by the following claims.

1. A method of treating or preventing a disease or disorder associated with premature aging in a subject comprising administering to the subject a composition comprising nicotinamide riboside.

2. The method of claim 1, wherein the composition further comprises pterostilbene.

3. The method of claim 1 or claim 2, wherein the disease or disorder associated with premature aging is Werner syndrome (WS), Bloom syndrome (BS), Rothmund-Thomson syndrome (RTS), Cockayne syndrome (CS), xeroderma pigmentosum (XP), trichothiodystrophy (TTD), combined xeroderma pigmentosum-Cockayne syndrome (XP-CS), trichothiodystrophy, or Hutchinson-Gilford progeria syndrome (HGPS).

4. The method of claim 3, wherein the disease or disorder associated with premature aging is Hutchinson-Gilford progeria syndrome (HGPS).

5. The method of claim 3, wherein the disease or disorder associated with premature aging is Werner syndrome (WS).

6. The method of claim 3, wherein the disease or disorder associated with premature aging is Cockayne syndrome (CS).

7. The method of claim 1 or claim 2, wherein the disease or disorder associated with premature aging is a disease or disorder caused by a mutation in a gene encoding a lamin protein.

8. The method of claim 7, wherein the lamin protein is lamin A.

9. The method of claim 7, wherein the lamin protein is lamin C.

10. The method of claim 1 or claim 2, wherein the disease or disorder associated with premature aging is a disease or disorder caused by a mutation in a gene encoding a DNA repair protein.

11. The method of claim 10, wherein the DNA repair protein is a RecQ protein-like helicases (RECQLs) or a nucleotide excision repair (NER) protein.

12. A method of treating or preventing a progeroid syndrome in a subject, comprising administering to the subject a composition comprising nicotinamide riboside.

13. The method of claim 12, wherein the composition further comprises pterostilbene.

14. The method of claim 12 or claim 13, wherein the progeroid syndrome is Werner syndrome (WS), Rothmund-Thomson syndrome (RTS), Cockayne syndrome (CS), trichothiodystrophy (TTD), combined xeroderma pigmentosum-Cockayne syndrome (XP-CS), trichothiodystrophy, or Hutchinson-Gilford progeria syndrome (HGPS).

15. The method of claim 12 or claim 13, wherein the progeroid syndrome is associated with or caused by a mutation in a gene encoding a lamin protein.

16. The method of claim 15, wherein the lamin protein is lamin A or lamin C.

17. The method of claim 12 or claim 13, wherein the progeroid syndrome associated with or caused by a mutation in a gene encoding a DNA repair protein.

18. The method of claim 17, wherein the DNA repair protein is a RecQ protein-like helicases (RECQLs) or a nucleotide excision repair (NER) protein.

19. A method of treating or preventing symptoms of a disease or disorder associated with premature aging in a subject in need thereof, comprising administering to the subject a composition comprising nicotinamide riboside.

20. The method of claim 19, wherein the composition further comprises pterostilbene.

21. The method of any one of claims 1 to 20, wherein the administration of the composition comprises administering one or more doses of the composition.

22. The method of claim 21, wherein each dose of the composition comprises at least 200 mg of nicotinamide riboside.

23. The method of claim 21, wherein each dose of the composition comprises at least 250 mg of nicotinamide riboside.

24. The method of claim 21, wherein each dose of the composition comprises at least 300 mg of nicotinamide riboside.

25. The method of claim 21, wherein each dose of the composition comprises at least 350 mg of nicotinamide riboside.

26. The method of claim 21, wherein each dose of the composition comprises at least 400 mg of nicotinamide riboside.

27. The method of claim 21, wherein each dose of the composition comprises at least 450 mg of nicotinamide riboside.

28. The method of claim 21, wherein each dose of the composition comprises at least 500 mg of nicotinamide riboside.

29. The method of claim 21, wherein each dose of the composition comprises at least 550 mg of nicotinamide riboside.

30. The method of any one of claims 21 to 29, wherein each dose of the composition comprises at least 15 mg of pterostilbene.

31. The method of any one of claims 21 to 29, wherein each dose of the composition comprises at least 25 mg of pterostilbene.

32. The method of any one of claims 21 to 29, wherein each dose of the composition comprises at least 50 mg of pterostilbene.

33. The method of any one of claims 21 to 29, wherein each dose of the composition comprises at least 75 mg of pterostilbene.

34. The method of any one of claims 21 to 29, wherein each dose of the composition comprises at least 100 mg of pterostilbene.

35. The method of any one of claims 21 to 29, wherein each dose of the composition comprises at least 125 mg of pterostilbene.

36. The method of any one of claims 21 to 29, wherein each dose of the composition comprises at least 150 mg of pterostilbene.

37. The method of any one of claims 21 to 36, wherein two or more doses of the composition are administered.

38. The method of any one of claims 21 to 37, wherein thirty or more doses of the composition are administered.

39. The method of any one of claims 21 to 38, wherein fifty or more doses of the composition are administered.

40. The method of any one of claims 21 to 39, wherein one hundred or more doses of the composition are administered.

41. The method of any one of claims 21 to 40, wherein the dose of the composition is administered at least once a week.

42. The method of any one of claims 21 to 40, wherein the dose is administered at least twice a week.

43. The method of any one of claims 21 to 40, wherein the dose is administered at least three times a week.

44. The method of any one of claims 21 to 40, wherein the dose is administered at least once a day.

45. The method of any one of claims 21 to 40, wherein the dose is administered at least twice a day.

46. The method of any one of claims 41 to 45, wherein the doses are administered for at least 7 days.

47. The method of any one of claims 41 to 45, wherein the doses are administered for at least 30 days.

48. The method of any one of claims 41 to 45, wherein the doses are administered for at least 60 days.

49. The method of any one of claims 41 to 45, wherein the doses are administered for at least 90 days.

50. The method of any one of claims 1 to 49, wherein the composition is formulated as a pill, a tablet, or a capsule.

51. The method of any one of claims 1 to 50, wherein the composition is administered orally.

52. The method of any one of claims 1 to 51, wherein the composition is self-administered.
